(19) United States
(12) Patent Application Publication
(10) Pub. No.: US 2026/0196357 A1
(43) Pub. Date: Jul. 9, 2026

(54) ASSESSING CARDIOVASCULAR DISEASE RISK USING RETINAL SCANS

(71) Applicant: Optain Health, Inc., Charlotte, NC (US)

(72) Inventors: Zongyuan Ge, Melbourne (AU); Mingguang He, Hong Kong (CN); Zhihong Lin, Melbourne (AU); Wei Meng, Melbourne (AU); Danli Shi, Hong Kong (CN)

(73) Assignee: Optain Health, Inc., Charlotte, NC (US)

(21) Appl. No.: 19/551,395

(22) Filed: Feb. 26, 2026

Related U.S. Application Data
(63) Continuation of application No. PCT/US2024/043753, filed on Aug. 23, 2024.
(60) Provisional application No. 63/579,689, filed on Aug. 30, 2023.

(51) Int. Cl. G16H 50/30; G16H 10/60; G16H 50/20

(52) U.S. Cl. CPC G16H 50/30; G16H 10/60; G16H 50/20

(57) ABSTRACT

The present disclosure relates to leveraging one or more machine-learning models to generate a cardiovascular risk score that corresponds to a prediction of a present or future presence or progression of cardiovascular disease for a given subject based on retinal scan data of the subject. The one or more machine-learning models may include (for example) a neural network-based feature generator configured to generate features by processing the retinal scan data and a prediction model that takes feature vectors and predict cardiovascular risk-factors as auxiliary target variables along with cardiovascular risk scores for a current and/or future point in time. The feature generator and the prediction model may be trained jointly in an end-to-end manner by computing a loss function that reduces the discrepancy between actual and predicted values and may apply weights to each predicted label based on its contribution to the prediction accuracy of cardiovascular risk scores.

### CROSS-REFERENCE TO RELATED APPLICATIONS

This application is a continuation of International Patent Application No. PCT/US2024/043753, filed on Aug. 23, 2024, which claims the priority to and the benefit of U.S. Provisional Application No. 63/579,689 filed on Aug. 30, 2023, which is hereby incorporated by reference in its entirety for all purposes.

### BACKGROUND

Globally, cardiovascular disease is estimated to be responsible for approximately one third of deaths. If an individual is diagnosed with cardiovascular disease, medication, behavioral changes, and/or procedures (e.g., to implant a stent in the heart or an artery; perform heart surgery, etc.) can be used to manage the disease and reduce the risk of death. However, determining any individual's risk for cardiovascular disease is not standardized and typically relies on disparate types of data. For example, one or more of the following types of tests may be relied upon: electrocardiogram, cardiac computerized tomography (CT), cardiac magnetic resonance imagining (MRI), ultrasound, blood work, stress tests, cardiac catheterization, and ultrasound. Results from these test(s) can be assessed alongside symptom identification, information about any family history of cardiovascular disease, and physician observations.

However, each of the above-listed tests have limitations that constrain the degree to which results of the test may be indicative of cardiovascular-disease existence, state, or risk. Further, many of these tests are selectively performed only when a specialist (which often follows a referral) has ordered the test. Thus, frequently, the tests may not be performed (e.g., as a result of a subject or medical provider not initiating them), which may result in cardiovascular disease being undiagnosed and untreated. Additionally, although cardiovascular disease accounts for a substantial portion of deaths across the world, a much smaller portion of people have cardiovascular disease (or diagnosed cardiovascular disease) at any given point in time. Thus, training data sets are often imbalanced and can result in false-negative biases. Further, obtaining a data set that can be used to train a machine-learning model to predict long-term diagnosis of or progression of cardiovascular disease can be particularly costly and difficult to obtain, as it may involve tracking subjects across a long time period. Many subjects and/or corresponding medical providers may, over time, cease to partake in (or prescribe) tests needed for long-term monitoring and/or intervening events may thwart obtaining such data points.

### SUMMARY

Certain aspects of the present disclosure relate to leveraging one or more machine-learning models to process retinal scan data for predicting a likelihood of a subject experiencing (presently or in a future time period) one or more cardiovascular diseases. The likelihood of the subject experiencing the one or more cardiovascular diseases may be represented as a cardiovascular risk score. The retinal scan data may include or may be generated based on one or more retinal scans (e.g., a retinal scan image of a retina of a left eye of the subject and/or a right eye of the subject). Each retinal scan image may depict all or part of the retina and/or all or part of one or more other parts of the eye e.g., optic disc, optic nerve, blood vessel, etc. The retinal scan may be preprocessed by applying various preprocessing techniques, such as normalization, scaling, color enhancements, augmentation, rotation, translation, or flipping, which may improve consistency and/or enhance representations of features of interest. By leveraging a first machine-learning model, a set of numeric features can be generated based on the retinal scan, where the first machine-learning may be (for example) a neural network configured to generate and process one or more features from the retinal scan to support subsequent generation of multiple labels. The set of numeric features (e.g., which may be aggregated to form a feature vector) may be processed by a second machine-learning model that may generate multiple labels including one or more other labels. The first machine-learning model and the second machine-learning may be trained jointly in an end-to-end manner as a single machine-learning model.

In some instances, the only input data to the machine-learning model is the retinal scan data. The multiple labels may represent and/or include (for example) the cardiovascular risk score representing a predicted risk of the subject experiencing one or more specific cardiovascular disease (e.g., a heart attack or stroke) within a defined time period. The risk score may represent (for example) a predicted probability that the subject currently has a cardiovascular disease (e.g., generally or of a specific type), a predicted severity of any cardiovascular disease of the subject, a predicted probability that the subject will have a cardiovascular disease (e.g., of any type or of a specific type) within a defined time period, and/or a predicted severity of a cardiovascular disease of the subject at a given future time point. The multiple labels may further include predicting one or more other labels that may be termed as cardiovascular-risk contributing factors, such as a label that predicts a retinal age gap characterizing a difference between an age predicted based on a retinal scan of a subject (a “predicted retinal age”) and an actual biological age.

The one or more other labels may predict a demographic characteristic (e.g., biological age, gender), diabetic status, body mass index, blood pressure and/or a current or past behavioral characteristic (e.g., alcohol drinking, smoking or physical activity) of the subject. In some instances, the prediction of the one or more other labels may be based on the retinal scan data. For example, a label that is used to predict a risk of the subject experiencing a cardiovascular disease may include a label defined to be a prediction of an age of a subject, where the prediction is made using the retinal scan data. In some instances, true data corresponding to the one or more other labels is accessed via (for example) an electronic health record, input from a subject, input from a care provider, etc. This accessed information can facilitate training the first machine-learning model and the second machine-learning model.

In some instances, the second machine-learning model may be an ordinal regression model trained using a loss function that prioritizes accuracy of the one or more first disease labels (e.g., over or instead of accuracy of the one or more other labels). During training, the ordinal regression model predicts the outputs by converting an underlying latent variable associated with a label into two or more ordinal classes that correspond to numeric ranges. The ordinal regression model then learns the probabilities of latent variable falling below each threshold and determines the likely ordinal class for each range. For example, the underlying latent variable for an integer age may be transformed into ordinal values by evaluating a set of thresholds—each corresponding to a different age bracket and then learns the probability of lying below each threshold. After the multiple labels are generated by the ordinal regression model, the labels may be weighted, and the weights can be used while training the machine-learning models and/or processing subsequent input data. For example, a first label relating to an existence or severity of a current or future cardiovascular disease or event may be weighted higher than another label (e.g., relating to a demographic characteristic, behavioral characteristic, etc.). To illustrate, a first label that predicts whether a subject will experience a cardiovascular event of a given type (e.g., stroke or heart attack or a fatal cardiovascular incident within a given time period, such as ten years) may be weighted more highly than each other label (e.g., whether the subject is male, and that the subject is between 20-30 years old, etc.).

One or more additional characteristics of the subject may further be used (e.g., addition to the retinal scan data) by the first and/or second machine-learning model. The one or more additional characteristics may include data that is present in and/or can be derived from (for example) an electronic health record or input provided by a subject. For example, the one or more additional characteristics may include and/or may be limited to data that is determined using one or more of: medical records, medical-provider observation, and/or subject input. The predicted multiple labels representing cardiovascular risk score of a subject and one or more other labels may be displayed in a graphical user interface (GUI).

In some aspects, a system is provided that includes one or more data processors and a non-transitory computer readable storage medium containing instructions which, when executed on the one or more data processors, cause the one or more data processors to perform part or all of one or more methods disclosed herein.

In some aspects, a computer-program product tangibly embodied in a non-transitory machine-readable storage medium, including instructions configured to cause one or more data processors to perform part or all of one or more methods or processes disclosed herein.

In some aspects, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by aspects and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present disclosure is described in conjunction with the appended figures:

FIG. 1 shows an exemplary overview of the present disclosure for predicting cardiovascular risk scores.

FIG. 2 illustrates a block diagram of a Cox regression model for estimating the cardiovascular risk scores at one or more points in time, in accordance with some aspects of the present disclosure.

FIG. 3A illustrates an exemplary network for predicting one or more labels from a retinal scan.

FIG. 3B illustrates one or more additional components of the exemplary network illustrated in the FIG. 3A.

FIG. 4A illustrates a block diagram of a network taking retinal scan data to generate feature vectors in accordance with some aspects of the present disclosure.

FIG. 4B illustrates one or more additional components of the network illustrated in the FIG. 4A.

FIG. 5 shows an illustrative example of a first graphical user interface (GUI) displaying prediction results for the retinal scan data of a subject.

FIG. 6 shows an illustrative example of a second GUI displaying trends of cardiovascular risk scores predicted by the techniques, as disclosed herein, for the retinal scan data of the subject.

FIG. 7 illustrates an exemplary workflow to predict cardiovascular risk scores using the retinal scan data in accordance with some aspects of the present disclosure.

FIG. 8 illustrates an exemplary block diagram of a computing system in which various aspects of the disclosed techniques may be executed.

### DETAILED DESCRIPTION

Some aspects of the present disclosure relate to leveraging one or more machine-learning (ML) models to generate a cardiovascular risk score that corresponds to a prediction of a present or future presence or progression of cardiovascular disease for a given subject based on retinal scan data of the subject. The retinal scan data can include part or all of one or more retina scans and/or may be derived based on one or more retinal scans. A retinal scan (such as fundus images, optical coherence tomography (OCT), retinal angiography) may depict part or the interior of an eye, including part or all of the retina, optic disc, optic nerve and blood vessels. The techniques, as disclosed herein, may include a machine-learning model comprising a first machine-learning model—a neural network-based feature generator and a second machine-learning model—a prediction model, where both the models are trained jointly in an end-to-end manner. The feature generator may be configured to generate a set of features by processing retinal scan data that includes all or part of the retinal scan and/or a preprocessed version thereof. The machine-learning model may transform the retinal scan data into the set of features (that may be aggregated to form a feature vector) by leveraging the feature generator and predict multiple labels by leveraging the prediction model that processes the feature vector. The multiple labels may include cardiovascular risk scores for a current and/or future point in time along with one or more other labels as auxiliary targets. The machine-learning model may be trained by computing a loss function that reduces the discrepancy between actual and predicted values and may apply weights to each predicted label based on its contribution to the prediction accuracy of cardiovascular risk scores.

The cardiovascular risk scores may represent (for example) a predicted risk of the subject experiencing one or more specific cardiovascular events (e.g., a heart attack or stroke) within a defined time period. Alternatively, the cardiovascular risk scores may represent (for example) a predicted probability or a likelihood that the subject currently has a cardiovascular disease (e.g., generally or of a specific type), a predicted severity of any cardiovascular disease of the subject, a predicted probability that the subject will have a cardiovascular disease (e.g., of any type or of a specific type) within a defined time period, and/or a predicted severity of a cardiovascular disease of the subject at a given future time point (e.g., in five or ten years). The one or more other labels of the multiple labels may identify one or more other characteristics of the subject. The one or more other characteristics of the subject may include a demographic characteristic (e.g., a subject's age, a subject's biological sex, etc.) and/or a current or past behavioral characteristic (e.g., whether the subject is or was a smoker, whether and/or a degree to which the subject drinks or historically drank alcohol, etc.). In some instances, the one or more other characteristics include a characteristic of a subject that may be referred as being a risk-factor characteristic for cardiovascular disease (e.g., whether the subject is or was a smoker, whether the subject has high blood pressure, a subject's age or age bracket, a subject's biological sex, diabetes status, total cholesterol (TC), or body mass index (BMI), etc.). The one or more other labels of the multiple labels can include a retinal age gap, which is the difference between a predicted age determined based on processing the retinal scan and the subject's actual age.

In some examples of training the machine-learning model, a single observation may include a retinal scan (from one eye or both eyes) as input, and multiple labels including cardiovascular risk scores at current and/or future points, one or more other labels (e.g., demographic data or risk-factors as output. However, tracking the subjects to collect long-term data for training the machine-learning model and to validate the cardiovascular risk predictions at current or future points may be impractical due to extensive resources involving cost and time. To address this limitation, various techniques may be used for labeling the dataset that estimates these cardiovascular risk scores for given time intervals from historical data of the subjects. These techniques may include, for example, Cox proportional hazard model, logistic regression, temporal deep learning models (e.g., recurrent neural networks), accelerated failure time models and parametric survival methods predicting the probability of disease onset over time based on risk-factors. In some instances, Cox model may be leveraged for modeling time-to-event data, such as time until a cardiovascular event occurs or the hazard (or risk) of an event occurring at any given time, given one or more characteristics.

After collecting and labeling data, the machine-learning model may be trained with retinal scan data as input and the outcomes may be the cardiovascular risk scores along with one or more other labels as the auxiliary targets. The retinal scan data may be processed by the feature generator (e.g., a pre-trained convolutional neural network (CNN) or vision transformer) to generate features that may be fed to a prediction model including multiple ordinal regression models, each making a different prediction. The ordinal regression model can predict different types of outcomes, also termed as ordinal outcomes, i.e., continuous (e.g., cardiovascular risk scores, age) and discrete (e.g., smoking status, gender, presence of a cardiovascular incident or disease) by modeling the relationship between predictors (features from feature generator) and an ordinal outcome. The ordinal regression model assumes an underlying latent variable associated with each outcome and segments this variable into ordinal classes by defining thresholds or cut-off points. For example, if the outcome is discrete, such as cardiovascular severity categorized as low, medium and high, the ordinal regression model estimates thresholds on the underlying latent variable that separate these classes. Similarly, if the outcome is continuous such as risk scores or age, the model may estimate how different age values affect underlying latent variable, which may be divided into thresholds corresponding to ordinal classes.

By calculating the probability of the latent variable falling below these thresholds, the model may learn and determine the likely ordinal class for each age range associated with cut-off points. This approach may allow ordinal regression to handle ordered ordinal categories while providing insights into both continuous and discrete predictions based on the same set of predictors. Following the ordinal estimation method, an ordinal cross-entropy loss may be computed for each ordinal model based on the predicted logits and true labels. A feature ranking module may use this loss to compute weights and adjust or rank the predicted risk-factors or feature vectors based on the contribution of individual risk-factor to the cardiovascular risk score prediction accuracy. The predicted cardiovascular risk scores may be displayed in a graphical user interface (GUI) along with auxiliary targets such as risk-factors, retinal age gap (difference between estimated retinal age based on retinal scan and actual biological age) indicating cardiovascular risks (i.e., higher the value higher the risk), and probability of occurring a cardiovascular incident (e.g., heart stroke or heart failure) at some point in time (e.g., 3 or 10 years).

The risk-factors and cardiovascular risk scores for both eyes may provide similar or overlapping information, regardless of whether the prediction is based on the left or right eye scan. In some embodiments, the potential redundancy can be utilized during training to enhance the accuracy of predictions, as any inconsistencies between the two can be used to detect and correct errors. Thus, a processing technique may begin with training of two separate “network branches” that correspond to the right and left eyes, and the results generated for the network branches may be subsequently assessed (during the processing technique) in combination. It will be appreciated that the processing for the separate network branches may be performed at least partly concurrently or at separate times), and results may be generated for each of the network branches. In some aspects, each of a pair of retinal scans from the left and right eyes are separately processed such that each of the scans are processed in the same manner. For example, for each network branch, each scan may be processed using a same feature-generation technique, features may be ranked or weighted using a same or similar manner, and/or a same or similar prediction model may be used to transform each scan (and/or corresponding features) into a prediction pertaining to cardiovascular disease during the training phase.

In some instances, at least some parameters used by the feature generator, prediction model and feature ranking of one network branch may be shared with the corresponding components of the other network branch, resulting in identical network branches. This may support learning uniform representation across both network branches generating respective predictions by computing the respective loss function. The feature ranking may be performed to prioritize predicting a degree to which each of the features contributes to improving an accuracy of the prediction pertaining to cardiovascular risk. During training, the processing technique may dynamically compare the losses from both network branches to identify which network branch has a higher loss, indicating less accurate predictions. This comparison may determine which network branch (including feature generator, prediction model and feature ranking) is performing better. The network branch with better performance may become a teacher network guiding the other network. Based on the results of the loss comparison, the features may be adjusted by computing gradients with respect to the teacher network to influence the updates of the network with higher-loss during backpropagation. It should be appreciated that the processing technique involving two separate network branches is for training purpose and since at least some parameters are shared between respective components included in both network branches, the networks may be identical to each other. Therefore, components from either of the network branches including feature generator, prediction model and feature ranking may be used for inference regardless of the input given from left eye or right eye.

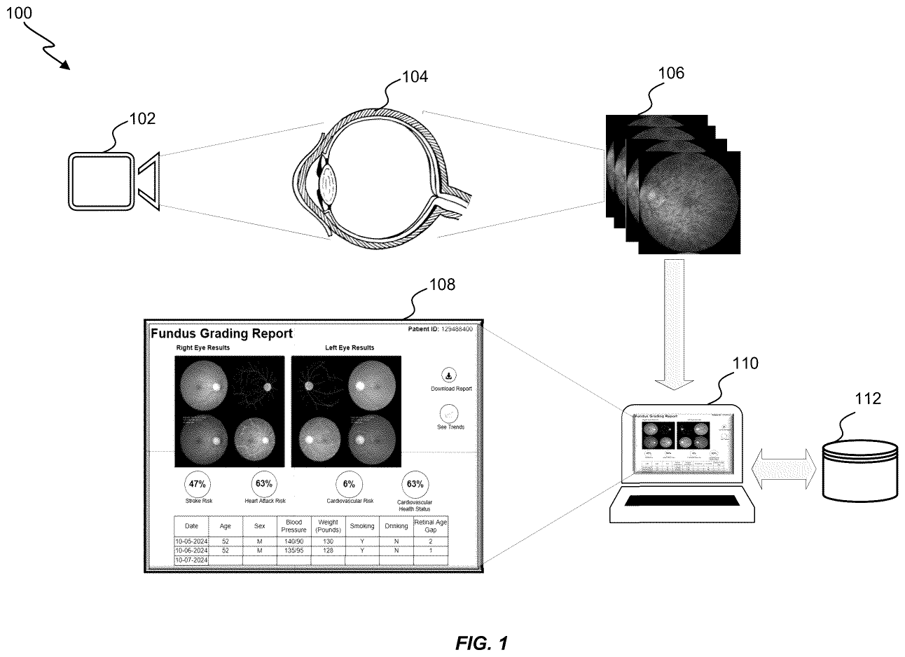

FIG. 1 shows an exemplary overview 100 of current disclosure for predicting cardiovascular risk score and one or more other labels. Exemplary overview 100 may include a retinal scan camera 102, a computing system 110 and a database 112. The retinal scan camera may be used to capture retinal scan data 106 that can include part or all of one or more retina scans and/or may be derived based on one or more retinal scans. A retinal scan may include an optical coherence tomography (OCT), an OCT angiography, a retinal fundus photography, an autofluorescence imaging or a scanning laser ophthalmoscopy of an eye 104. The retinal scan is a non-invasive and painless procedure, therefore safe for both physicians and patients in treatment or detection of any underlying conditions. Retinal scans, for example, fundus images may capture features such as vessel diameter, tortuosity, and signs of retinopathy, while OCT may provide insights into retinal thickness and macular volume and retinal angiography may highlight blood flow and vascular integrity. These anatomical features may reflect systemic health conditions linked to cardiovascular risks that the machine-learning model, as disclosed herein, may correlate retinal abnormalities with known risk-factors to predict cardiovascular risk scores.

The retinal scanning, particularly fundus photography (a medical ocular imaging technique) may be used to capture specialized two-dimensional detailed photographs of the interior surface of the eye, including the retina, optic disc, optic nerves and blood vessels, fovea, macula, choroid and/or vitreous humor. In some embodiments, an output of the retinal scan (e.g., a fundus image) may be used to facilitate diagnosing and/or monitoring an ocular condition (e.g., diabetic retinopathy, glaucoma or age-related macular degeneration) and/or a systematic condition (a cardiovascular disorder, metabolic disorder, or infectious disease). To capture retinal scan data 106, the retinal scan camera 102 may be focused along the subject's pupillary axis to aim the light at the pupil for illumination and then adjust the lens to find a good focus and avoid light reflections. A single fundus image of a non-dilated eye may capture less than 450 of the back of the eye. Typically, several images of the eyes are taken while guiding the subject to look up, down, left and right to create a larger field of view of the retina.

The retinal scan data 106 may be processed by a computing system 110 that may include a user device, computer, laptop or a specialized device that may have the retinal scan camera 102 embedded into it. The computing system 110 may take retinal scan data 106 through a communication network that may include a wired (e.g., Ethernet, USB (universal serial bus) or fiber optics) or wireless media (e.g., Wi-Fi, Bluetooth or cellular networks) and store these in its own memory or a database 112 that may be built within the computing system or on a cloud-based storage system. While the data is illustrated as being stored at a single location, it should be appreciated that this is not intended to be limiting—the data may be stored in multiple memories or locations. The computing system 110 may be where the computations are carried out including the computation of the techniques disclosed herein that may help in determining various cardiovascular disease risk-factors. It may also generate a risk score that corresponds to a prediction of a present or future presence or progression of cardiovascular disease for a given subject based on retinal scan data 106 of the subject taken from retinal scan camera 102. The results may be shown in a graphical user interface (GUI) as a retinal scan report 108 showing various prediction results.

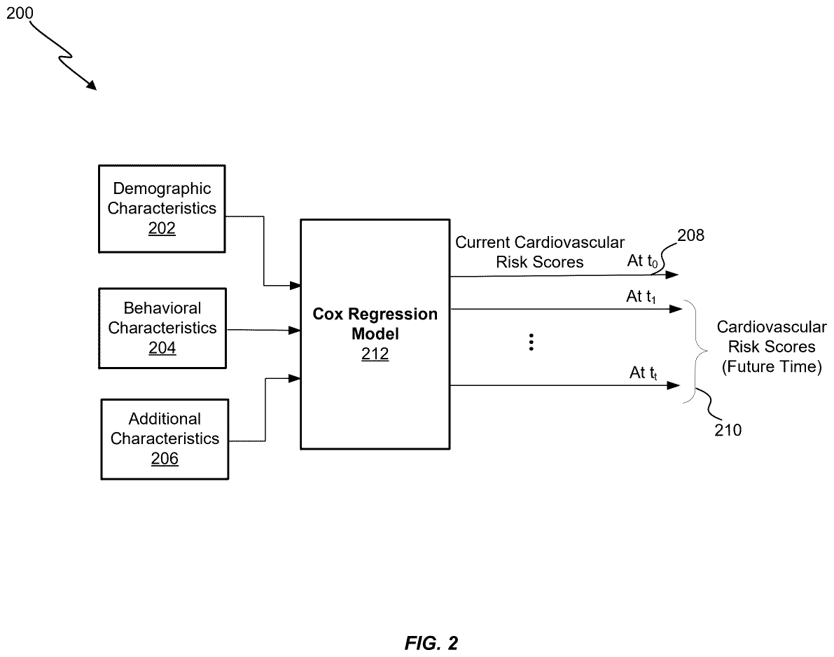

FIG. 2 illustrates a block diagram 200 of a Cox regression model 212 for estimating cardiovascular risk scores at one or more points in time, in accordance with some aspects of the present disclosure. Collecting long-term data for training a machine-learning or deep learning model to predict cardiovascular risk at some current or particular future time point may be a difficult and expensive procedure. Moreover, using actual cardiovascular diseases or incidents (e.g., heart attacks, strokes or coronary artery disease events) as training targets may be impractical because of long-term tracking to observe whether the subject developed cardiovascular diseases and imbalanced data that in turn may result in false-negative biases. This long-term follow-up may be resource intensive involving high costs and longer delays in gathering enough training data and perform model validation effectively. To address this, Cox proportional hazard model, also termed as Cox model 212, may be used for estimating cardiovascular risk scores (e.g., generally or of a specific type) based on the one or more other characteristics (e.g., demographic and/or behavioral characteristics) and/or one or more additional characteristics associated with the subject.

For example, the one or more other characteristics of the subject may include a demographic characteristic 202 (e.g., a subject's age, a subject's biological sex, etc.) and/or a current or past behavioral characteristic 204 (e.g., whether the subject is or was a smoker, whether and/or a degree to which the subject drinks or historically drank alcohol, etc.). The one or more other characteristics may further include a characteristic of a subject that may be referred as being a risk-factor characteristic for cardiovascular disease (e.g., whether the subject is or was a smoker, whether the subject has high blood pressure, a subject's age or age bracket, a subject's biological sex, diabetes status, total cholesterol (TC), retinal age gap, or body mass index (BMI), etc.).

The one or more additional characteristics 206 of the subject (represented in conjunction with the retinal data in the input data) can include data points that are readily available via (for example) an electronic health record (EHR) (providing e.g., blood tests or other health conditions), medical-provider observations (e.g., notes during medical visits or clinical assessments), or input provided by a subject (e.g., self-reported symptoms or lifestyle factors such as engagement in physical activities. EHR may also provide subject demographics, clinical history, lab results and medication records such as history of medical diagnosis. By collecting these characteristics, current cardiovascular risk scores 208 and/or future cardiovascular risk scores (e.g., at t₁, . . . , tₜ) 210 can be estimated using the Cox model 212 for obtaining labeled training data. These scores can be paired with retinal scan data 106 to create labeled dataset for training the machine-learning model. This approach can leverage existing data, making data collection and labelling quicker, and cost effective as compared to long-term tracking. It should be appreciated that EHR data may be stored in a cloud-based database.

Cox model 212 is a regression model that may be used to examine the relation between the survival time of subjects and one or more predictor variables. It may be used for modeling time-to-event data, such as time until a cardiovascular event occurs. The Cox model 212 may estimate the hazard (or risk) of an event occurring at any given time, given certain characteristics. An assumption may be made in Cox model 212 that the hazard function for an individual is a product of a baseline hazard function and a function of the characteristics of the individual (predictor variables). The Cox model 212 may give output for a current cardiovascular risk score 208 and cardiovascular risk scores at one or more future points in time (e.g., at t₁, . . . , tₜ) 210 for a cardiovascular incident or a specific cardiovascular disease. The predictor variables (or one or more characteristics), may include e.g., blood pressure, age, gender, diabetes status etc. The hazard function at time t for an individual with total p predictor variables or characteristics, C=(C₁, C₂, . . . Cₚ) may be given as, h(t|C)=h₀(t) exp (B₁C₁+B₂C₂+ . . . +BₚCₚ), where h(t|C) is the hazard function at time t for a given set of characteristics C, h₀(t) is the baseline hazard function representing the hazard for an individual with all predictor variables set to 0, and β₁+β₂+ . . . +βₚ are the coefficients representing effect of each predictor variable on the hazard. Using historical data where the time to a cardiovascular incident or event and the predictor variables are known, the coefficients may be estimated.

To predict current cardiovascular risk scores 208 at time to for an unknown individual, values of the predictor variables associated with the unknown individual may be input to the Cox model. Additionally, the risk of experiencing a cardiovascular event within a future time period (e.g., next 5 or 10 years) can be calculated by integrating the hazard function over the time period t to get the cumulative hazard as,

$${\int }_{0}^{t} h ( s \vert C ) ds ,$$

where s is the variable of integration representing time that ranges from 0→t. The survival function that represents the probability of not experiencing the event by time t may be computed as, S(t|C)=exp(−H(t|C)). Finally, the probability of experiencing the event within the time period t may be calculated from the formula: P(event by the time t|C)=1−S(t|C).

Additionally, the current and future cardiovascular risks using one or more characteristics or predictor variables may be estimated using various other models and techniques. For example, logistic regression can estimate the likelihood of current cardiovascular risk, while parametric survival models and accelerated failure time models can predict the probability of disease onset over time based on risk-factors. Machine-learning methods such as random forest, gradient boosting machines and deep learning models such as recurrent neural networks (RNN) or its variants can capture complex patterns and predict risks based on historical and evolving data. Moreover, established risk score calculators such as Framingham risk score and QRISK can also be adapted to estimate current and future risks. These approaches may leverage relevant characteristics (demographic and/or behavioral) for reliable risk predictions through validations and calibrations. These predicted risk scores (current or future) may be used for labeling the dataset for further processing by the techniques as disclosed herein.

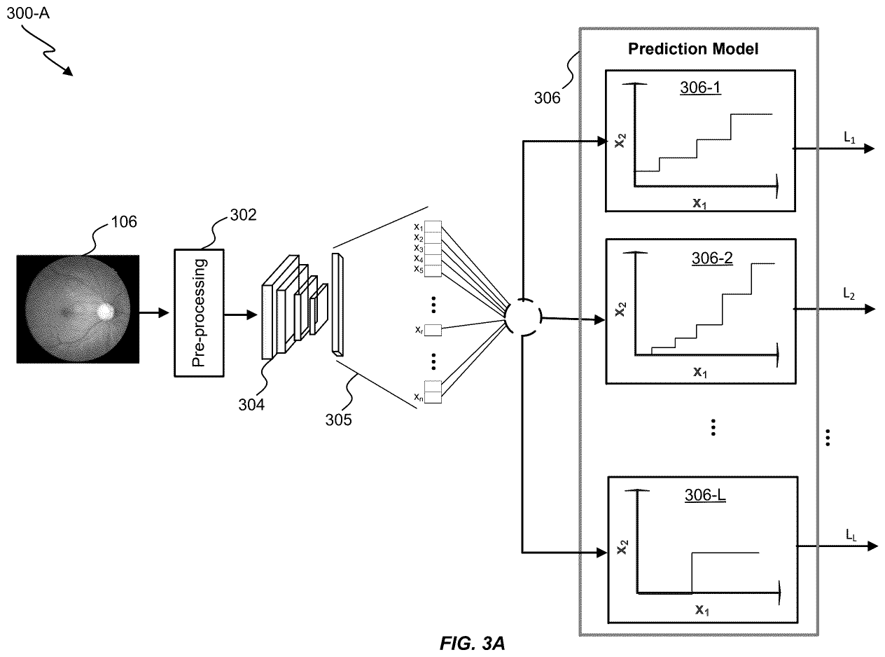

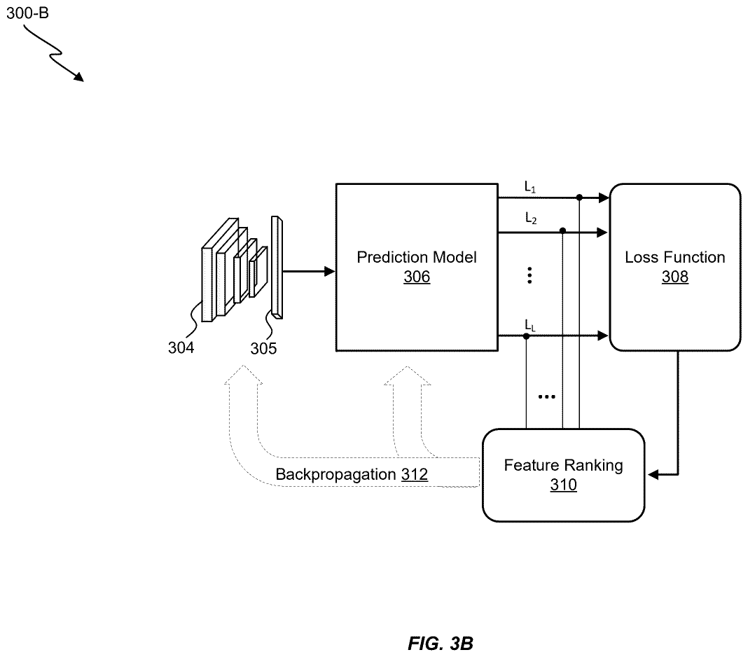

FIG. 3A illustrates an exemplary network 300-A for predicting multiple labels from a retinal scan. The network 300-A may take the retinal scan 106 as input that may be preprocessed for a consistent format and to highlight certain regions of interest for subsequent analysis. The preprocessing 302 may involve, for example, normalization, resizing or scaling, cropping to extract region of interest (ROI), contrast enhancement, and applying filters such as noise reduction or other filters to highlight various aspects such as blood vessels to predict cardiovascular risks or identifying and isolating optic disc region for age-related prediction. Preprocessing 302 may also include augmenting data for making network robust and invariant to the spatial orientation within the input images so that the network generalizes better. Preprocessing 302 may include, for example, rotation, flipping, translation, masking, color jitter, grayscale, zooming in/out or adding noise may also increase size of the training dataset and improve model robustness. The preprocessed image may be passed through a first machine-learning model to generate features from the retinal scan data 106 for identifying patterns in the image that are relevant to prediction of multiple labels.

The aim of the first machine-learning model, also termed hereinafter as feature generator 304, may be to effectively convert retinal scan data 106 into meaningful features for predicting multiple labels. The multiple labels may include a disease label that may predict (for example) whether the subject currently has cardiovascular disease (e.g., generally or of a specific type), whether the subject currently will have cardiovascular disease (e.g., generally or of a specific type) at a particular future time point (e.g., in one year, five years, ten years, etc.), a severity of any current cardiovascular disease (e.g., generally or a severity of a specific type of cardiovascular disease) of the subject, a severity at a particular future time point of any cardiovascular disease of the subject. The multiple labels may further include subject characteristics (or risk-factors) e.g., age or age bracket, gender, systolic blood pressure (SBP), diabetes status, total cholesterol (TC), or body mass index (BMI), etc., past or current behavioral characteristics (e.g., whether the subject is or was a smoker, and/or whether a degree to which the subject drinks or historically drank alcohol, etc.), and/or a retinal age gap, which characterizes a difference between a predicted age based on processing the retinal scan of the subject and the actual biological age. A higher value of retinal age gap may also be an indicator of cardiovascular health deterioration that may lead to a cardiovascular risk.

For generating features, feature generator 304 may include convolutional neural network (CNN) or pre-trained CNNs such as Alexnet, VGGNet, ResNet (residual networks), GoogLeNet or DenseNet by replacing, adding or removing final layers to generate feature vectors as output. These modified models may then be fine-tuned on the retinal scan data 106 to adapt the pre-trained features for effective feature generation task. Specialized architectures may also be designed and adapted for retinal imaging including attention U-net that incorporates attention mechanisms to focus on relevant regions of the image or DeepLab that leverages atrous convolutions for dense features extraction and improved segmentation. Additionally, other suitable networks such as recurrent neural network (RNN), long short-term memory (LSTM), hybrid networks (e.g., combining CNN-RNN), and vision transformers (ViT) may offer powerful alternatives to CNNs for feature generation. Vision transformer may generate patch embeddings (typically corresponding to fix-sized non-overlapping patches of the input image) along with positional embeddings and pass through multiple transformer layers comprising multi-head self-attention mechanisms and feed-forward neural networks capturing long-range dependencies and relationships between patches. The output from the transformer layers can be used as feature vectors, which may then be passed to a second machine-learning model, hereinafter as prediction model 306.

The task of prediction model 306 may be to predict multiple labels of various types e.g., one or more regression labels (y_{rgr}) such as age, BMI, or cardiovascular risk scores and categorical labels (y_{ctg}) such as gender, smoking or drinking status, and/or severity of risk (low, mild, severe). The training set T including n training samples (i.e., feature vectors x) for a total L multiple labels may be denoted as,

$$T = {\{ {x}_{i} , ( {y}_{i}^{rgr} , {y}_{i}^{ctg} ) \}}_{i = 1}^{n} , ( rgr = 1 , … , p ; ctg = 1 , … , q ; p + q = L ) ,$$

where xᵢ denotes a feature vector 305 or retinal scan data 106 of an ith training sample,

$${y}_{i}^{rgr}$$

denotes the associated regression labels, and

$${y}_{i}^{ctg}$$

denotes the associated categorical labels. The prediction model 306 may be defined as the function of f_{θ}: x→(y_{ctg},y_{rgr}). In this prediction model 306, ordinal regression models may be used to predict these multiple labels where a label is divided into classes using a set of thresholds holding a natural order but the intervals between the classes may not be equal. The prediction model 306 may include one regression model for each label of total L labels i.e., 306-1, 306-2, . . . , 306-L. For example, age may be predicted by ordinal regression model 306-1 that may divide the underlying latent variable into different intervals such as 1-10, 11-20, 21-30, 31-45 etc.

Instead of directly predicting a label, the ordinal regression model may output logits, z=[z₁, z₂, . . . , zₖ₋₁] for each boundary between k classes within a label. Each logit z represents log-odds of the input belonging to a class greater than a specific threshold. These logits may be used to determine cumulative probabilities that represent the likelihood of the input falling into each ordinal class i.e.,

$$P ( Y \leq j \vert x ) = σ ( {z}_{j} ) = \frac{1}{1 + {e}^{- {z}_{j}}} .$$

The predicted interval may be determined by finding the highest cumulative probability against the set of intervals that exceeds a certain threshold. For example, a feature vector (x) 305 given to the prediction model 306 for the age brackets mentioned above, may output logits for each threshold as z₁, z₂, z₃, z₄ and cumulative probabilities e.g., P(Y≤(0-10)|x)=σ(z₁), P(Y≤(11-20)|x)=σ(z₂) and soon. The class with maximum cumulative probability exceeding the threshold is chosen as predicted age. Binary outcomes such as gender or drinking status may be treated as special cases with two classes.

FIG. 3B illustrates one or more additional components for the network illustrated in the FIG. 3A. It will be appreciated that the first machine-learning model (i.e., feature generator 304) and the second machine-learning model i.e., prediction model 306 can collectively serve as a single model and can be trained together in an end-to-end manner. The ordinal cross-entropy loss may be used as loss function 308 to train this combined model 300-A for estimating multiple labels. This loss function may calculate a discrepancy between the predicted and actual categories or values. In some examples, after computing loss, the predicted labels from the prediction model 306 may be fed to a feature ranking 310, which may apply weights wᵢ to each label (i→L) based on the loss values, and the weights can be used while training the model and/or processing subsequent input data. For example, a first label relating to an existence or severity of a current or future cardiovascular disease or event may be weighted higher than one or more other labels (e.g., relating to a demographic characteristic, behavioral characteristic, etc.).

To illustrate, a first label that predicts whether a subject will experience a cardiovascular event of a given type (e.g., stroke or heart attack or a fatal cardiovascular incident within a given time period, such as ten years) may be weighted more highly than each other label (e.g., whether the subject is male, and that the subject is between 20-30 years old, etc.). Using the cumulative probabilities, the ordinal cross-entropy loss for an ordinal regression output (or label) i=1→L with Kᵢ ordinal classes may be estimated as:

$$Loss = {\sum }_{i = 1}^{L} {w}_{i} [ {\sum }_{k = 1}^{{K}_{i} - 1} ( ( {y}_{i} \leq k ) log {p}_{i , k} ( x ) ) + ( {y}_{i} > k ) log ( 1 - {p}_{i , k} ( x ) ) ] .$$

In this loss, p_{i,k}(x) represents predicted cumulative probability for the ith label and kth class given the input x that may be computed as: p_{i,k}(x)=σ(θ_{i,k}−fᵢ(x)), where θ_{i,k} is a threshold for kth class within ith ordinal regression outcome. Alternatively, the feature vectors from the feature generator 304 may be fed to the feature ranking 310 that based on the loss function 308 may rank each feature i.e., x_{Adjusted}=x·w. For example, if a feature contributes to a higher loss, it may be down-weighted, while features contributing to lower losses may be up-weighted by the feature ranking module 310. Feature ranking 310 may be performed by various techniques including augmenting an additional layer to the feature generator 304 or prediction model 306, post-processing techniques such as permutation importance, training a separate model or using attention mechanism to determine feature significance. During backpropagation 312, gradients of the loss with respect to model parameters (i.e., weights and biases of each model e.g., feature generator 304 and each regression model 306-1, . . . 306-L) may be computed that may flow backward to the prediction model 306 and feature generator 304 updating weights of each individual model.

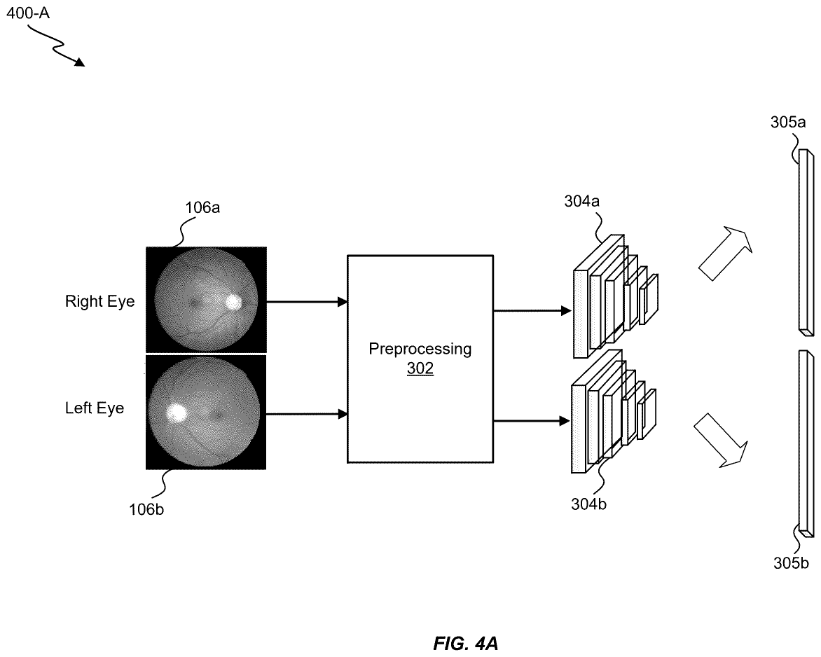

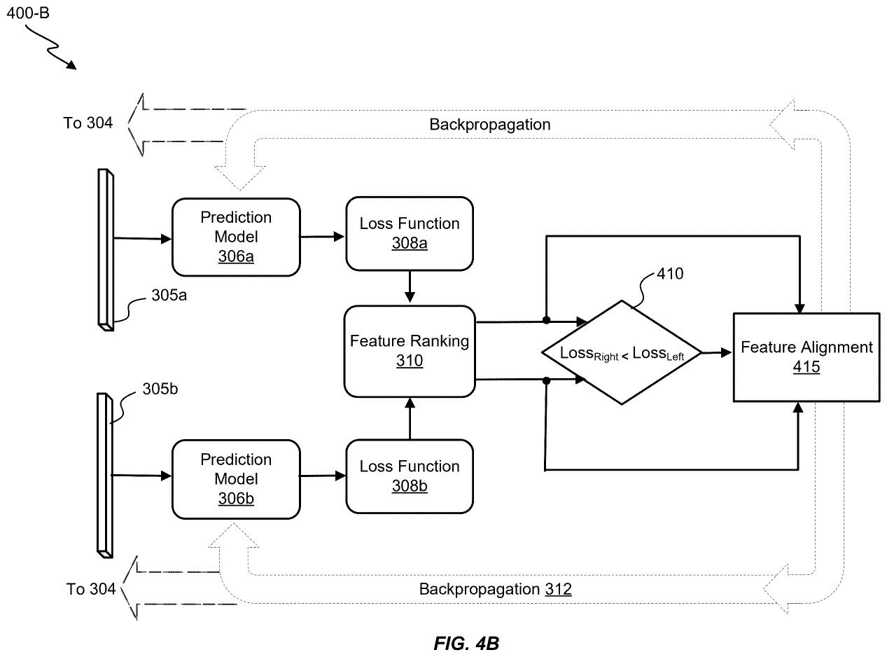

The one or more other characteristics (e.g., age, diabetic status, behavioral characteristics or additional characteristics) and cardiovascular risk scores for both eyes may yield consistent values or overlapping information regardless of whether the prediction is made from left or right retinal scan. This potential redundancy can be exploited during training phase to improve reliability of predictions, as discrepancies between the two can be used to identify and correct errors. Additionally, by training the network on a diverse dataset including retinal images from both eyes, the model may learn to generalize across different individuals and reduce overfitting. Therefore, the exemplary architecture 400 illustrated in the FIG. 4 may be designed structuring two separate network branches that correspond to right and left retinal scans (i.e., 106a and 106b, respectively) for improving consistency and robustness in determining cardiovascular risk scores. It will be appreciated that the network branches may perform processing at least partly concurrently or at separate times, where each scan of the two network branches may be processed in a similar manner.

For example, an exemplary network 400-A may take right and left retinal scan data i.e., 106a and 106b during the training phase structuring two network branches, where after preprocessing 302 each retinal scan may be processed for generating respective feature vectors 305a and 305b by using a same feature generation technique (i.e., 304a and 304b) that may share at least some or all parameters (weights and biases). The parameter sharing across both network branches may structure identical networks that enable consistency and lesser space (as fewer parameters are used). FIG. 4B illustrates additional components of the exemplary network 400-A in the FIG. 4A. The feature vectors 305a and 305b may be individually directed into respective same or similar prediction techniques 306a and 306b to transform each scan (and/or corresponding features) into a prediction pertaining to cardiovascular risk (or cardiovascular disease risk). Depending on the specific implementation, these prediction techniques (i.e., 306a and 306b) may share parameters enabling uniform learning across both branches (or instead operate independently allowing independent learning for the prediction models).

In both network branches, the predicted labels may be evaluated by computing respective loss functions 308a and 308b for each network branch to measure error between actual values and predicted values. For example, the loss function for right eye branch 308a given a right eye feature vector or right eye retinal scan (x_{right}) 305a may be computed as:

$${Loss}_{right} = {\sum }_{i = 1}^{L} {w}_{i} [ {\sum }_{k = 1}^{{K}_{i} - 1} ( ( {y}_{i} \leq k ) log {p}_{i , k} ( {x}_{right} ) ) + ( {y}_{i} > k ) log ( 1 - {p}_{i , k} ( {x}_{left} ) ) ] ,$$

and similarly for left eye,

$${Loss}_{left} = {\sum }_{i = 1}^{L} {w}_{i} [ {\sum }_{k = 1}^{{K}_{i} - 1} ( ( {y}_{i} \leq k ) log {p}_{i , k} ( {x}_{left} ) ) + ( {y}_{i} > k ) log ( 1 - {p}_{i , k} ( {x}_{left} ) ) ] .$$

The feature ranking 310 may evaluate the importance of different features and/or predicted labels contributing to prediction accuracy and assign weights accordingly. At 410, the exemplary architecture 400 may dynamically compare the loss from both network branches to determine which branch incurs a higher loss, indicating less accurate predictions. This comparative analysis may identify the network branch performing better than the other and can subsequently guide the other network branch.

The feature alignment 415 may be activated based on the outcome of the loss comparison at 410, for example, if loss from the right network branch Loss_{right} is greater than the loss from left network branch Loss_{left}, or vice versa, alignment process may be initiated. The network branch with superior performance or lower loss is identified as a teacher network branch representing more accurate and reliable features. During training, feature alignment 415 may adjust the features by computing gradients of the components (e.g., 304, 306 and/or 310) from the teacher network branch to influence the updates of the higher-loss branch during backpropagation. This approach may help feature generators 304a and 304b and the prediction models 306a and 306b to learn more accurate representation, facilitating knowledge transfer and improving the predictive accuracy of the underperforming branch. At least some or all respective components in both the network branches may share parameters resulting in identical networks with same or similar parameters, for example, right feature generator 304a may share weights with left feature generator 304b. Similarly, right prediction model 306a may share weights with left prediction model 306b. Thus, any component of either of two network branches may be used for inference tasks, thereby generating consistent results for both retinal scans.

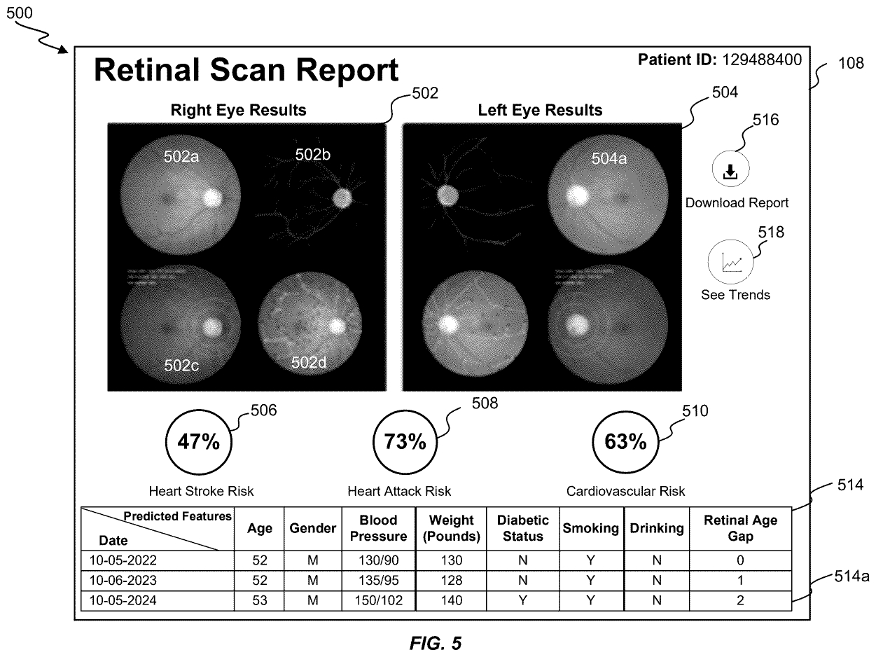

FIG. 5 shows an illustrative example of a first graphical user interface (GUI) 500 (also referred previously as retinal scan report 108), displaying prediction results, in accordance with one or more techniques as disclosed herein, for retinal scan data 106 of a subject. The first GUI 500 shows a retinal scan report 108 that may include different visualizations of left eye and right eye. For example, right eye results 502 may include a right fundus image 502a, a vascular fundus image 502b highlighting blood vessels, 502c highlighting optic disc in a fundus image and 502d highlighting other anatomical structures e.g., fovea. Similar results may also be seen in FIG. 5 for the left eye in 504. The first GUI 500 may further include the cardiovascular risk score 510 that may show a probability of the subject developing cardiovascular disease within a specified time frame (e.g., current or after 3 years). The cardiovascular risk score 510 may be transformed in percentage to represent an approximation of a probable risk that the subject may suffer from cardiovascular disease or incident in the present or in future. It may further include one or more cardiovascular disease risks such as heart stroke risk 506 and heart attack risk 508 for the time points in future e.g., after 3 years.

In the bottom of the illustrative example of the first GUI 500, a table 514 may be shown that may display the predicted one or more other labels (or risk-factors) of multiple labels that may be predicted by the techniques, as disclosed herein, as auxiliary targets. A single row of the table 514 includes predicted one or more other labels e.g., age, gender, blood pressure, weight, diabetic status, smoking status, drinking status for the retinal scan data (i.e., 502a and/or 504a) taken (or performed) at a given date in numeric format i.e., “DD-MM-YYYY”. Table 514 may further include the retinal age gap of the subject that is an indirect marker to cardiovascular risk 510 with high value indicating more risk. In this illustrative example, the likelihood of the subject experiencing the one or more cardiovascular diseases presently may be represented as a cardiovascular risk score 510. It can be seen that the predicted one or more other labels in the table 514 also validate the cardiovascular risk 510. For example, from the retinal scan the subject is experiencing presently cardiovascular risk of 63% (indicating severity level—moderate). The associated one or more labels in row 514a of table 514 depicts that the subject may have started smoking, developing diabetes and increased blood pressure. The retinal age gap has also increased (as compared to previous years) that also serves as an indicator encouraging proactive measures to reduce overall cardiovascular risk.

On the right side of the illustrative example of the first GUI 500, there may be a “download report” option 516 to store a local copy of the retinal scan report 108, this may give functionality to the user to share the downloaded report or send it to a physician. Other options may also be provided e.g., to see the trends of past risk scores (if subject's history is available) and future predicted risk scores estimated by the techniques as disclosed herein of retinal scans of the subject.

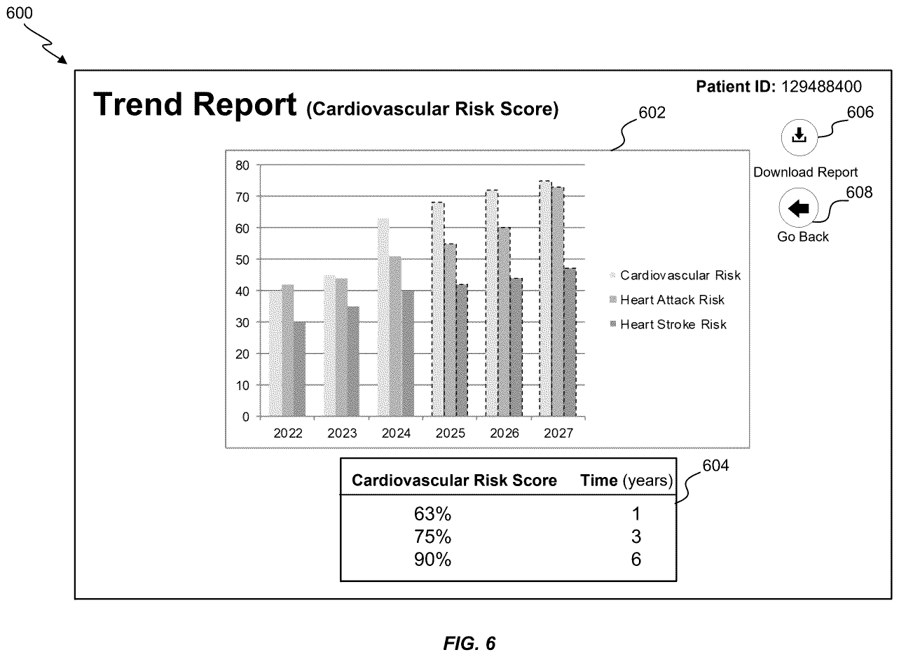

FIG. 6 shows an illustrative example of a second graphical user interface (GUI) 600 displaying yearly trends of cardiovascular risk scores. The past cardiovascular risk scores may be obtained by the techniques, as disclosed herein, by inputting (manually) the retinal scan data 106 taken at that instant or extracted from an EHR. The cardiovascular risk trend 602 may include trends of the predicted cardiovascular risk scores 510 and cardiovascular risk scores (e.g., heart attack 508 and heart stroke 506) taken for each year. The cardiovascular risk trend 602 in FIG. 6 shows an increased risk of cardiovascular disease for this particular subject. In the bottom of the second GUI 600, a table 604 may be shown that may include predicted cardiovascular risk scores 510 of the subject generated by the disclosed techniques over the years e.g., current and next 3 years, in a tabular form. On the right side of the illustrative example of the second GUI 600, there may be an option to download the cardiovascular risk score trends from the computing system 110, this may give functionality to the user to share the downloaded report or send to a physician. There may be an option to go back to the retinal scan report 108 by clicking the go back button 608. The retinal scan report 108 and cardiovascular risk score trends from the second GUI 600 in combination may give the user complete information about the cardiovascular disease risk score, cardiovascular health score and various cardiovascular disease risks (heart attack or stroke).

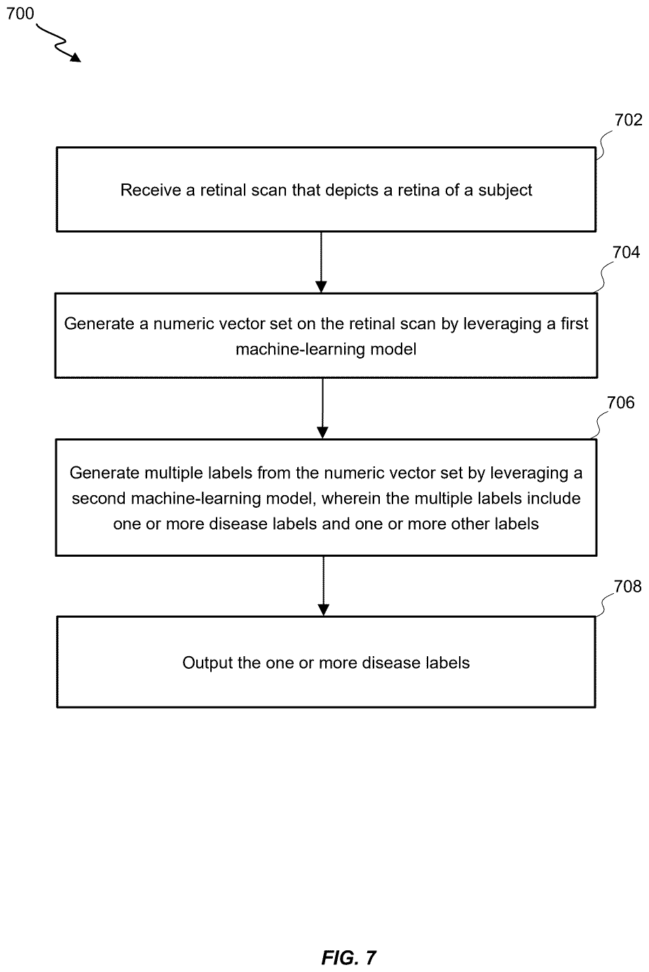

FIG. 7 illustrates an exemplary workflow to predict cardiovascular risk 510 using the retinal scan data 106 in accordance with some aspects of the present disclosure. The blocks in workflow 700 are illustrated in a specific order, while the order can be modified, for example, some blocks may be performed before other, and some blocks may be performed simultaneously. The block can be performed by hardware, software, or a combination thereof. For detecting cardiovascular disease risk, a process at block 702 may include obtaining retinal scan data 106 using the retinal scan camera 102 from one eye 104 (or both eyes) of a subject. The computing system 110 may then receive the retinal scan data 106 from the retinal scan camera 102. Block 704 may include processing the retinal scan data 106 to extract a numeric vector by leveraging a first machine-learning model, also termed herein as feature generator 304. This may compress the retinal scan data 106 for the second machine-learning model, also termed herein as prediction model 406. In some instances, the first machine-learning model may include a deep learning model like a neural network, such as a convolutional neural network, a long short-term memory (LSTM) model or a transformer model to transform a retinal scan data 106 into a set of numeric features 305. The numeric features or feature vector 305 may then be fed to the second machine-learning model for further processing.

At block 706, multiple labels may be generated from the numeric vector (or feature vector) 305 using a second machine-learning model that may be an ordinal regression model. Multiple labels may include one or more disease labels that predict, with respect to a current time or a future time, or a severity of the cardiovascular disease and one or more other labels (e.g., demographic or behavioral characteristics). Some aspects may generate a risk score that may correspond to a prediction of a present or future presence of or progression of cardiovascular disease for a given subject based on retinal scan data 106 of the subject. At block 708, one or more disease labels may be output pertaining to the cardiovascular risk 510. The risk score may represent (for example) a predicted risk of the subject experiencing one or more specific cardiovascular events (e.g., a heart attack or stroke) within a defined time period. The risk score may represent (for example) a predicted probability that the subject currently has a cardiovascular disease (e.g., generally or of a specific type), a predicted severity of any cardiovascular disease of the subject, a predicted probability that the subject will have a cardiovascular disease (e.g., of any type or of a specific type) within a defined time period, and/or a predicted severity of a cardiovascular disease of the subject at a given future time point.

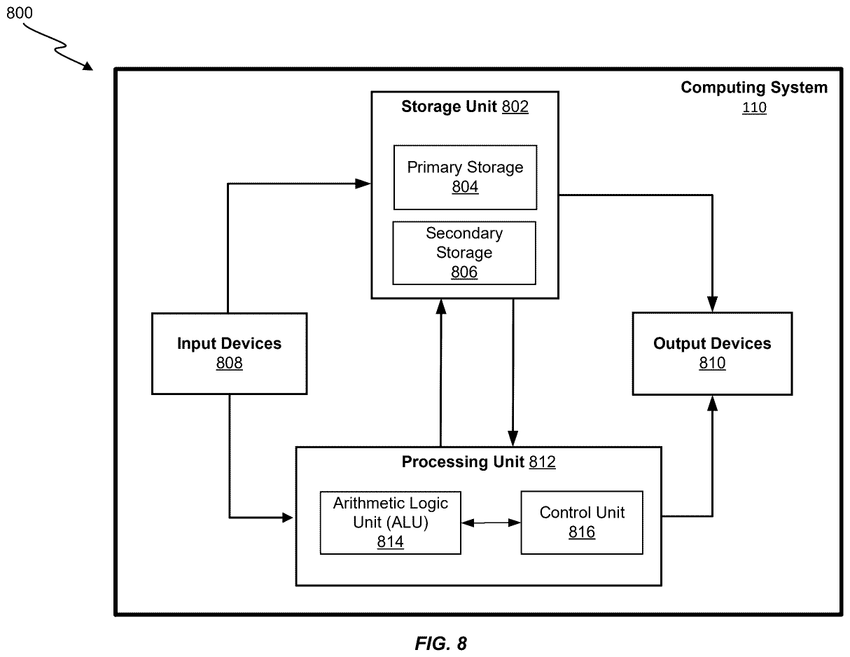

FIG. 8 is an example illustration of a computing system 800, (or interchangeably 110 from the FIG. 1), in which various embodiments of the present disclosure may be implemented. The functionality described herein can be performed, at least in part or a combination of one or more hardware or software logic components. For example, the techniques described above for assessing cardiovascular risk for a subject using retinal scans by leveraging machine-learning models can be implemented in computer-executable instructions. The instructions can be executed by processing unit 812 that may be a combination of an arithmetic logic unit 814 that performs arithmetic and logical operations, and a control unit 816 that may help in execution of the instructions. The control unit 816 may direct and coordinate the operation of the processor with other parts of the computer by synchronizing data flow between different components of the processing unit 812. It may manage the flow of instructions and data between various components. The control unit 816 may decode an operation code (opcode) and may convert them into control signals to coordinate how data moves within the processing unit 812. The control unit 816 may regulate execution units such as the arithmetic logic unit 814 and the flow of data to primary storage 804 and secondary storage 806.

To provide additional context for various aspects thereof, FIG. 8 and the following description are intended to provide a brief, general description of the computing system in which the various aspects can be implemented. While the description above is in the general context of computer-executable instructions that can run on one or more computing systems, those skilled in the art will recognize that a novel implementation also can be realized in combination with other program modules and/or as a combination of hardware and software. The computing system for implementing various aspects includes a processing unit 812 having one or more processors (also referred to as microprocessors), a computer-readable storage medium (where the medium is any physical device or material on which data can be electronically and/or optically stored and retrieved) such as a data storage unit 802 (computer readable storage medium/media also include magnetic disks, optical disks, solid state drives, external memory systems, and flash memory drives), and a system bus. The data storage unit 802 may have a primary storage 804 and a secondary storage 806 as described here in. The primary storage 804 and the secondary storage 806 may differ in speed of access, connection with the computer's processor and data retrieval speeds. Primary storage 804 may often be directly connected to the computer's processor, boasts rapid data retrieval speeds. In contrast, secondary storage 806 may be designed for long-term storage and may have slower access times.

The computing system 800 can include various microprocessors, such as single-processor, multi-processor, single-core, and multi-core units for processing and storage. Additionally, experts in the field recognize that the innovative system and methods can be applied to other computing configurations, including minicomputers, mainframe computers, personal computers (such as desktops, laptops, and tablet PCs), handheld computing devices, microprocessor-based consumer electronics, and similar systems. These systems can be interconnected with one or more associated devices.

In some aspects, the computing system 800 can include one of several computers employed in a datacenter and/or computing resources (hardware and/or software) in support of cloud computing services for portable and/or mobile computing systems such as wireless communications devices, cellular telephones, and other mobile-capable devices. Cloud computing services, include, but are not limited to, infrastructure as a service (IaaS), platform as a service, software as a service (SaaS), storage as a service (StaaS), data as a service (DaaS), security as a service and APIs (application program interfaces) as a service. In some instances, data storage unit 802 can include computer-readable storage (physical storage) medium such as a volatile memory (e.g. random-access memory (RAM) also termed as the primary storage 804) and a non-volatile memory (e.g., (ROM)). A basic input/output system (BIOS) can be stored in the non-volatile memory and includes the basic routines that facilitate the communication of data and signals between components within the computing system, such as during startup. The volatile memory also includes a high-speed RAM such as static RAM for caching data.

As an illustrative example (without limiting the scope), the data storage unit 802 may include program modules. These modules can encompass client applications, web browsers, mid-tier applications, relational database management systems (RDBMS), and more. Additionally, the storage unit 802 holds program data and an operating system. The operating system running can include (for example) Microsoft Windows®, Apple Macintosh®, or Linux. Furthermore, commercially available UNIX®-like operating systems (such as GNU/Linux variants and Google Chrome OS) and mobile operating systems (like iOS, Windows® Phone, Android OS, BlackBerry® OS, and Palm® OS) are part of this landscape. Notably, portions of the operating system, program modules, and program data can be cached in the storage unit 802—both volatile memory (e.g., RAM) and non-volatile memory (e.g., ROM). This flexibility allows the disclosed architecture to be implemented using a variety of commercially available operating systems or combinations thereof (including virtual machines).

In some other examples, the computing system 800 may have additional features or functionality. For example, the computing system 800 may also include additional data storage devices (removable and/or non-removable) such as, for example, magnetic disks, optical disks, or tape. Computer-readable media may include, at least, two types of computer-readable media, namely computer storage media and communication media. Computer storage media may include volatile and non-volatile, removable, and non-removable media implemented in any method or technology for storage of information, such as computer readable instructions, data structures, program modules, or other data.

The storage media of the computing system 800 may also include removable storage, and non-removable storage. EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store the targeted information and which computing system 800 can access are examples of computer storage media in addition to RAM and ROM. Additionally, the computer-readable media might have computer-executable instructions that the processing unit 812 can use to carry out the different tasks and/or operations mentioned in this article. In contrast, communication media may embody computer readable instructions, data structures, program modules, or other data in a modulated data signal, such as a carrier wave, or other transmission mechanism.

One or more input devices, such as a keyboard, mouse, pen, voice input device, touch input device, etc., may also be included in the computing system 800. There might also be one or more output devices 810, including speakers, printers, displays, and so on. These devices are not covered in detail here because they are well known in the field. To establish communication, the computing system 800 may further have one or more network interfaces. This would enable the computing system 800 to communicate with other systems or devices, for example, over a network. Both wired and wireless networks could be a part of these networks. Here, the computing system 800 is one example of a suitable device or system and is not intended to suggest any limitation as to the scope of use or functionality of the various embodiments described.

Other well-known computer environments, configurations, and/or systems that may be appropriate for use with the embodiments include, but are not limited to, network PCs, mainframe computers, programmable consumer electronics, set top boxes, game consoles, programmable consumer electronics, personal computers, server computers, hand-held or laptop devices, multiprocessor systems, microprocessor-based systems, and/or the like. For instance, part or all the computing system 800 components could be put into use in a cloud computing environment, where resources and/or services are made available for user devices to consume on a selected basis via a computer network.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation, such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claim is:

1. A computer-implemented method comprising:
receiving a retinal scan that depicts a retina of a subject;
generating a numeric vector set of the retinal scan by leveraging a first machine-learning model;
generating multiple labels by processing the numeric vector set by leveraging a second machine-learning model, wherein the first machine-learning model is trained jointly with the second machine-learning model, and wherein the multiple labels include:
one or more disease labels that predict, with respect to a current time or a future time point, whether the subject has or will have a cardiovascular disease or a severity of the cardiovascular disease; and
one or more other labels; and
outputting the one or more disease labels.

2. The computer-implemented method of claim 1, wherein the first machine-learning model is a neural network.

3. The computer-implemented method of claim 1, wherein the second machine-learning model is an ordinal regression model.

4. The computer-implemented method of claim 1, wherein the second machine-learning model was trained using a loss function that prioritizes accuracy of the one or more disease labels over accuracy of the one or more other labels.

5. The computer-implemented method of claim 1, wherein the multiple labels include a label corresponding to a variable that is a risk-factor for developing the cardiovascular disease within a defined time period.

6. The computer-implemented method of claim 1, wherein the one or more other labels include a label that predicts a retinal age gap that indicates a difference between a predicted age of the subject determined based on processing the retinal scan and an actual age of the subject.

7. The computer-implemented method of claim 1, wherein the one or more other labels include a label that predicts a demographic characteristic and a current or past behavioral characteristic of the subject.

8. A system comprising:
one or more data processors; and
a non-transitory computer readable storage medium containing instruction which, when executed on the one or more data processors, cause the one or more data processors to perform a set of operations including:
receive a retinal scan that depicts a retina of a subject;
generate a numeric vector set of the retinal scan by leveraging a first machine-learning model,
generating multiple labels by processing the numeric vector set by leveraging a second machine-learning model, wherein the multiple labels include:
one or more disease labels that predict, with respect to a current time or a future time point, whether the subject has or will have a cardiovascular disease or a severity of the cardiovascular disease; and
one or more other labels; and
outputting the one or more disease labels.

9. The system of claim 8, wherein the first machine-learning model is a neural network.

10. The system of claim 8, wherein the second machine-learning model is an ordinal regression model.

11. The system of claim 8, wherein the second machine-learning model was trained using a loss function that prioritizes accuracy of the one or more disease labels over accuracy of the one or more other labels.

12. The system of claim 8, wherein the one or more other labels includes a label corresponding to a variable that is a risk-factor for developing the cardiovascular disease within a defined time period.

13. The system of claim 8, wherein the one or more other labels include:
a label that predicts a retinal age gap that indicates a difference between a predicted age of the subject determined based on processing the retinal scan and an actual age of the subject; and
a label that predicts a demographic characteristic and a current or past behavioral characteristic of the subject.

14. A computer-program product tangibly embodied in a non-transitory machine-readable storage medium, including instructions configured to cause one or more data processors to perform to perform a set of operations comprising:
receiving a retinal scan that depicts a retina of a subject;
generating a numeric vector set of the retinal scan by leveraging a first machine-learning model;
generating multiple labels by processing the numeric vector set by leveraging a second machine-learning model, wherein the multiple labels include:
one or more disease labels that predict, with respect to a current time or a future time point, whether the subject has or will have a cardiovascular disease or a severity of the cardiovascular disease; and
one or more other labels; and
outputting the one or more disease labels.

15. The computer-program product of claim 14, wherein the first machine-learning model is a neural network.

16. The computer-program product of claim 14, wherein the second machine-learning model is an ordinal regression model.

17. The computer-program product of claim 14, wherein the second machine-learning model was trained using a loss function that prioritizes accuracy of the one or more disease labels over accuracy of the one or more other labels.

18. The computer-program product of claim 14, wherein the one or more other labels includes a label corresponding to a variable that is a risk-factor for developing the cardiovascular disease within a defined time period.

19. The computer-program product of claim 14, wherein the one or more other labels include a label that predicts a retinal age gap that indicates a difference between a predicted age of the subject determined based on processing the retinal scan and an actual age of the subject.

20. The computer-program product of claim 14, wherein the one or more other labels include a label that predicts a demographic characteristic and a current or past behavioral characteristic of the subject.
